(19) United States
(12) Patent Application Publication
(10) Pub. No.: US 2026/0355505 A1
(43) Pub. Date: Nov. 12, 2026

(54) Pelvic Floor Muscle Rehabilitation Device

(71) Applicant: Shenzhen Lute Jiacheng Supply Chain Management Co., Ltd., Shenzhen (CN)

(72) Inventors: Yanhong Li, Shenzhen (CN); Songlin Shen, Shenzhen (CN)

(21) Appl. No.: 19/712,260

(22) Filed: Jun. 18, 2026

(30) Foreign Application Priority Data
May 25, 2026 (CN) 202620749465.5

(51) Int. Cl. A61N 1/05

(52) U.S. Cl. CPC A61N 1/0524

(57) ABSTRACT

A pelvic floor muscle rehabilitation device, comprising an inner arm, an outer arm, and a connecting portion connecting the inner arm and the outer arm, the inner arm is provided with at least two stimulation electrodes and at least two detection electrodes, the stimulation electrodes and the detection electrodes being spaced apart along a circumferential direction of the inner arm; the inner arm has a first end proximate to the connecting portion and a second end distal from the connecting portion, and an outer cross-sectional diameter of the inner arm first increases and then decreases from the first end toward the second end, thereby forming a maximum outer diameter portion on the inner arm; the stimulation electrodes and the detection electrodes extend in a direction from the second end toward the first end and extend at least across the maximum outer diameter portion.

### CROSS-REFERENCE TO RELATED APPLICATIONS

The present application claims priority to Chinese Patent Application No. 202620749465.5, filed on May 25, 2026, the entire contents of which are incorporated herein by reference.

### TECHNICAL FIELD

The embodiments of the present application relate to the technical field of maternal and infant products, and in particular to a pelvic floor muscle rehabilitation device.

### BACKGROUND

The pelvic floor muscle rehabilitation device is a device configured to apply electrical stimulation to pelvic floor muscles to promote muscle contraction, thereby achieving pelvic floor muscle repair and functional enhancement. In the prior art, such devices typically employ a probe structure configured to be inserted transvaginally, with electrodes disposed on the surface of the probe for delivering electrical stimulation currents.

However, existing pelvic floor muscle rehabilitation devices commonly suffer from the following problems: after the probe is inserted into the vagina, due to the natural morphology of the vaginal wall and the presence of secretions, the electrodes often fail to make adequate and stable contact with the pelvic floor muscles, resulting in high contact resistance, which adversely affects the transmission efficiency of electrical stimulation signals and the therapeutic effect. Some products attempt to improve contact by increasing the overall size of the probe, but this significantly increases user discomfort and foreign body sensation. Other products form local protrusions on the probe surface to enhance contact, but because the probe's external shape is not effectively coordinated with the electrode positions, they still fail to achieve stable, low-impedance tissue contact at specific sites while ensuring insertion comfort.

Moreover, in existing devices, if stimulation of pelvic floor muscles at different circumferential locations within the vagina is desired, the user typically needs to manually rotate the probe to adjust the electrode orientation, which is inconvenient to operate and makes it difficult to simultaneously stimulate or detect multiple sites.

Therefore, how to enable the pelvic floor muscle rehabilitation device, after insertion, to not only ensure user comfort but also allow the electrodes to make stable and adequate contact with pelvic floor muscles at multiple circumferential locations without frequent manual adjustment is a technical problem urgently to be solved in the art.

### SUMMARY

The technical problem to be solved by the present application is the problem in existing pelvic floor muscle rehabilitation devices that it is difficult to achieve stable and adequate contact between the electrodes and the pelvic floor muscles at different circumferential locations within the vagina while ensuring insertion comfort.

To solve the above technical problem, the present utility model adopts the following technical solution: a pelvic floor muscle rehabilitation device, comprising an inner arm, an outer arm, and a connecting portion connecting the inner arm and the outer arm, wherein the inner arm is configured to be inserted into a vagina, and the outer arm is configured to be held by a user outside the vagina; wherein the inner arm is provided with at least two stimulation electrodes and at least two detection electrodes, the stimulation electrodes and the detection electrodes being spaced apart along a circumferential direction of the inner arm; the inner arm has a first end proximate to the connecting portion and a second end distal from the connecting portion, and an outer cross-sectional diameter of the inner arm first increases and then decreases from the first end toward the second end, thereby forming a maximum outer diameter portion on the inner arm; the stimulation electrodes and the detection electrodes extend in a direction from the second end toward the first end and extend at least across the maximum outer diameter portion.

In an optional embodiment, a total number of the stimulation electrodes and the detection electrodes is four, and the four electrodes are evenly spaced apart along the circumferential direction of the inner arm.

In an optional embodiment, the stimulation electrodes and the detection electrodes are alternately arranged along the circumferential direction of the inner arm.

In an optional embodiment, the two stimulation electrodes are arranged adjacent to each other, and the two detection electrodes are arranged adjacent to each other.

In an optional embodiment, the inner arm is substantially ellipsoidal in shape.

In an optional embodiment, each of the stimulation electrodes and the detection electrodes is an arc-shaped sheet electrode extending along a surface of the inner arm.

In an optional embodiment, the outer arm has an elongated shape to facilitate single-handed holding and adjustment of an orientation of the inner arm.

In an optional embodiment, the connecting portion is an arcuate bent section so that a predetermined angle is formed between the inner arm and the outer arm.

In an optional embodiment, the connecting portion is elongate and deformable by bending.

In an optional embodiment, the inner arm comprises a first frame and a first soft layer covering an outer surface of the first frame, a first accommodating space being formed inside the first frame; the pelvic floor muscle rehabilitation device further comprises a first circuit board disposed in the first accommodating space; the stimulation electrodes and the detection electrodes, as electrode pads, are disposed on an outer surface of the first frame and electrically connected to the first circuit board; the first soft layer is provided with openings, the electrode pads being at least partially exposed through the openings, and the first soft layer has a hardness less than a hardness of the first frame.

In an optional embodiment, in a direction from the first end to the second end, an outer diameter of the first frame first gradually increases and then gradually decreases, a circumferential outer surface of the first frame is provided with outwardly protruding protrusions, and the electrode pads cover the protrusions.

In an optional embodiment, each protrusion comprises an outwardly protruding top surface, each electrode pad comprises a main body portion, a shape of the main body portion matches a shape of the top surface of the protrusion, the main body portion covers the top surface of the protrusion, and the main body portion is exposed through the opening and protrudes beyond an outer surface of the first soft layer.

In an optional embodiment, each protrusion further comprises a side surface disposed around a periphery of the top surface of the protrusion, each electrode pad further comprises a folded edge connected to a periphery of the main body portion, and the folded edge is located on an inner side of the main body portion proximate to the protrusion, the folded edge covers the side surface of the protrusion, and an outer surface of the folded edge facing away from the protrusion is bonded to an inner sidewall of the opening.

In an optional embodiment, the top surface and the side surface of the protrusion are connected via a connecting curved surface, and the electrode pad further comprises a transition curved surface matching the connecting curved surface, the folded edge being connected to the periphery of the main body portion via the transition curved surface.

In an optional embodiment, the transition curved surface is at least partially exposed through the opening.

In an optional embodiment, a shortest distance between the periphery of the main body portion and an edge of the folded edge proximate to the main body portion ranges from 0.2 mm to 2 mm.

In an optional embodiment, an outer surface of the folded edge is provided with an infiltration portion recessed toward an inner surface of the folded edge, the infiltration portion being configured to receive an adhesive material.

In an optional embodiment, the infiltration portion comprises a plurality of infiltration holes, the plurality of infiltration holes being spaced apart along a circumferential direction of the folded edge; and/or the infiltration portion comprises an infiltration groove located on the folded edge at a position away from the periphery of the main body portion.

In an optional embodiment, an edge of each opening is lower than an outer surface of the first soft layer, the first soft layer comprises a transition bevel disposed around each opening, the transition bevel connects between the outer surface of the first soft layer and the edge of the opening, and in an exposure direction of the electrode pad, the transition bevel gradually moves away from a center of the opening.

In an optional embodiment, the first soft layer comprises a first region and a second region, the first region being closer to the opening than the second region, and the first region has an elastic modulus greater than that of the second region.

In an optional embodiment, the electrode pad has an elongate flat shape extending in a direction from the first end to the second end.

In an optional embodiment, the outer arm comprises a second frame, a second accommodating space being formed inside the second frame, and the connecting portion comprises a connecting wire; the pelvic floor muscle rehabilitation device further comprises a second circuit board disposed in the second accommodating space, the second circuit board being electrically connected to the first circuit board via the connecting wire to transmit control signals to the first circuit board.

In an optional embodiment, the outer arm further comprises a second soft layer covering an outer surface of the second frame, the connecting portion further comprises a third soft layer covering the connecting wire, the third soft layer is connected between the first soft layer and the second soft layer, and the first soft layer, the second soft layer, and the third soft layer form an integrally molded structure.

In an optional embodiment, the device further comprises a battery disposed in the first accommodating space and electrically connected to the first circuit board.

In an optional embodiment, a side of the inner arm opposite to the connecting portion is provided with the detection electrodes, and no stimulation electrode is disposed on said side of the inner arm.

By adopting the above technical solution, the present utility model has at least the following beneficial effects compared to the prior art: the embodiments of the present application, by configuring the outer cross-sectional diameter of the inner arm to first increase and then decrease from the first end toward the second end to form a maximum outer diameter portion, render the inner arm as a whole having an ellipsoidal shape that conforms to human physiological structure, which not only facilitates smooth and stable insertion of the inner arm into the vagina without damaging the vaginal mucosa, but also achieves good conformity with the pelvic floor muscles after insertion, significantly improving user comfort. Meanwhile, by arranging the stimulation electrodes and the detection electrodes spaced apart along the circumferential direction of the inner arm, and having each electrode extend in the direction from the second end toward the first end and cover at least the maximum outer diameter portion, after the inner arm is inserted into the vagina, the electrodes can simultaneously make adequate and stable contact with the pelvic floor muscles at different circumferential locations within the vagina, eliminating the need for the user to frequently manually rotate the device during treatment to adjust electrode orientation. This circumferential multi-directional electrode layout, together with the moderate expansion effect of the maximum outer diameter portion on the vaginal tissue, can reduce the contact resistance between the electrodes and the tissue, facilitate efficient transmission of electrical stimulation currents and accurate acquisition of myoelectric signals, thereby improving the repair effect and detection accuracy for the pelvic floor muscles.

### BRIEF DESCRIPTION OF THE DRAWINGS

To more clearly illustrate the technical solutions of the embodiments of the present application or the prior art, a brief introduction of the drawings used in the description of the embodiments or the prior art is provided below. It is apparent that the drawings described below only illustrate some embodiments of the present application. For those skilled in the art, other drawings may be obtained based on the structures shown in these drawings without any creative effort.

FIG. 1 is a schematic structural diagram of a pelvic floor muscle rehabilitation device according to an embodiment of the present application, from a first viewing angle.

FIG. 2 is a schematic diagram of a first electrode arrangement of the pelvic floor muscle rehabilitation device according to an embodiment of the present application, from a second viewing angle;

FIG. 3 is a schematic diagram of the first electrode arrangement of the pelvic floor muscle rehabilitation device according to an embodiment of the present application, from a third viewing angle;

FIG. 4 is a schematic diagram of a second electrode arrangement of the pelvic floor muscle rehabilitation device according to an embodiment of the present application, from the second viewing angle;

FIG. 5 is a schematic diagram of the second electrode arrangement of the pelvic floor muscle rehabilitation device according to an embodiment of the present application, from the third viewing angle;

FIG. 6 is an exploded structural diagram showing a soft layer of the pelvic floor muscle rehabilitation device and other parts of the pelvic floor muscle rehabilitation device according to an embodiment of the present application.

FIG. 7 is an exploded structural diagram of a first frame of the pelvic floor muscle rehabilitation device according to an embodiment of the present application.

FIG. 8 is a schematic structural diagram of the pelvic floor muscle rehabilitation device according to an embodiment of the present application, from a fourth viewing angle.

FIG. 9 is a schematic cross-sectional structural diagram of the pelvic floor muscle rehabilitation device according to an embodiment of the present application.

FIG. 10 is an enlarged view of a partial structure within the dashed box 90 of FIG. 9.

The achievement of the objectives, functional features, and advantages of the present application will be described in detail in combination with the embodiments and with reference to the accompanying drawings.

### DETAILED DESCRIPTION OF THE EMBODIMENTS

In the present application, the terms “arranged,” “provided with,” and “connected” should be understood broadly. For example, a connection may be fixed, detachable, or integrally constructed; it may be mechanical or electrical; it may be direct or indirectly connected through an intermediary, or it may refer to internal communication between two devices, components, or constituent parts. Those of ordinary skill in the art can understand the specific meanings of these terms in the context of the present application based on specific circumstances.

The terms “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “radial,” and “circumferential” are used to describe orientations or positional relationships based on the orientations or positional relationships shown in the accompanying drawings. These terms are used solely for the purpose of facilitating the description of the present application and simplifying the description, and do not indicate or imply that the referenced devices or components must have a specific orientation or be constructed and operated in a specific orientation. Therefore, these terms should not be construed as limiting the present application.

Furthermore, the terms “first” and “second” are used for descriptive purposes only and should not be construed as indicating or implying relative importance or implicitly specifying the quantity of the indicated technical features. Thus, features defined by “first” or “second” may explicitly or implicitly include at least one such feature. In the description of the present application, the term “plurality” means at least two, such as two, three, etc., unless explicitly stated otherwise.

Additionally, some of the above terms may be used to indicate meanings other than orientation or positional relationships. For example, the term “upper” may, in some cases, also be used to indicate a certain dependency or connection relationship. Those of ordinary skill in the art can understand the specific meanings of these terms in the context of the present application based on specific circumstances.

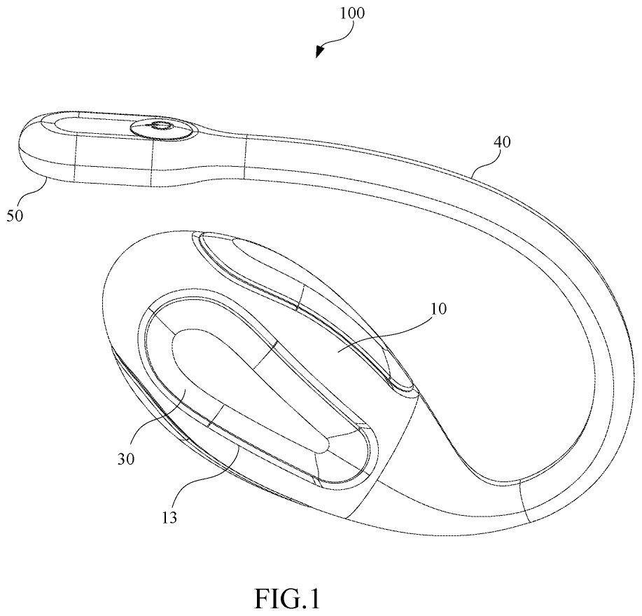

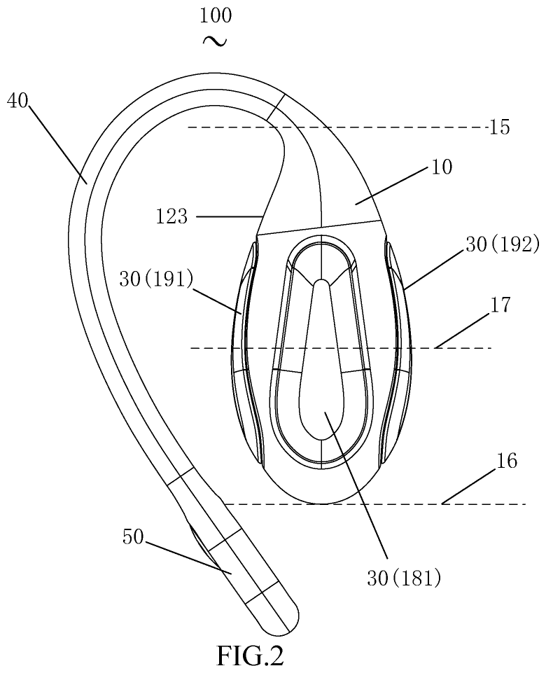

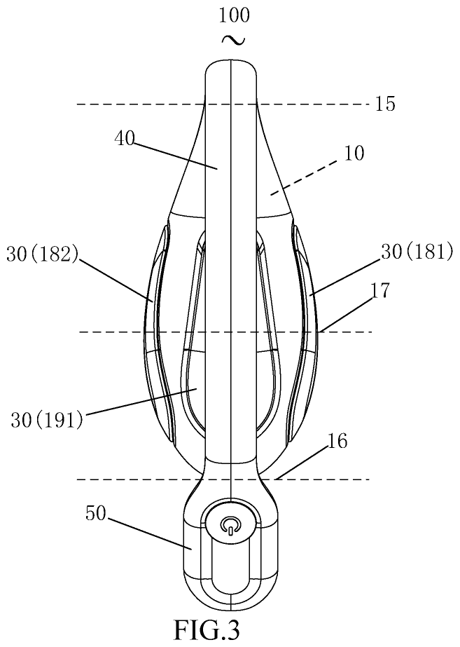

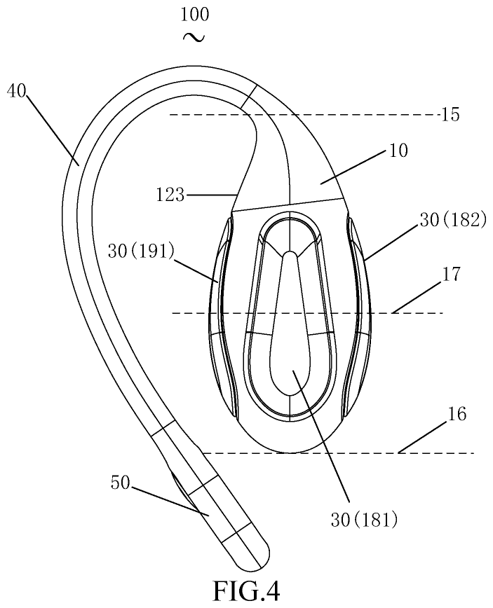

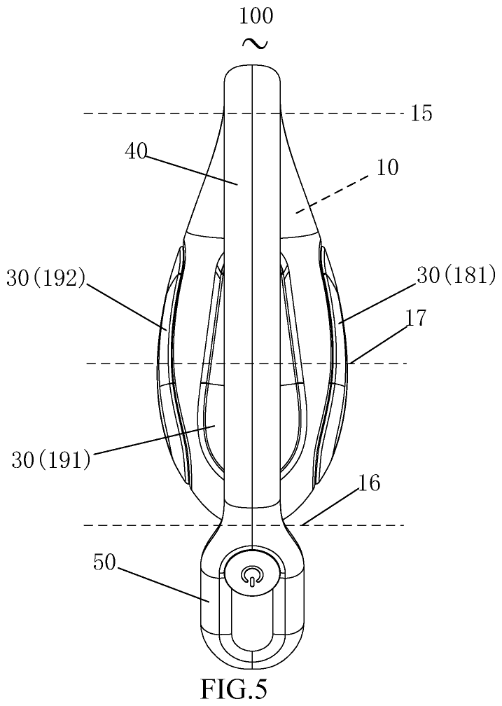

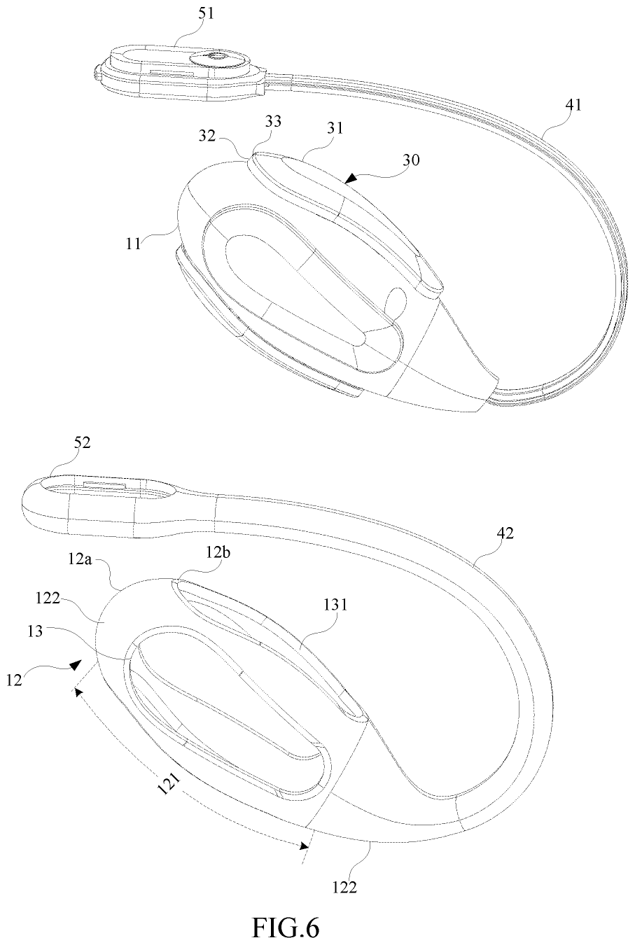

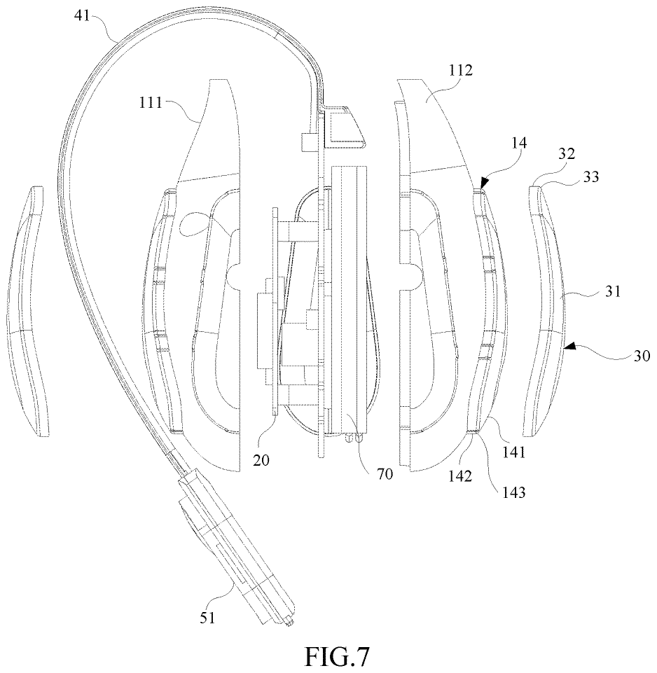

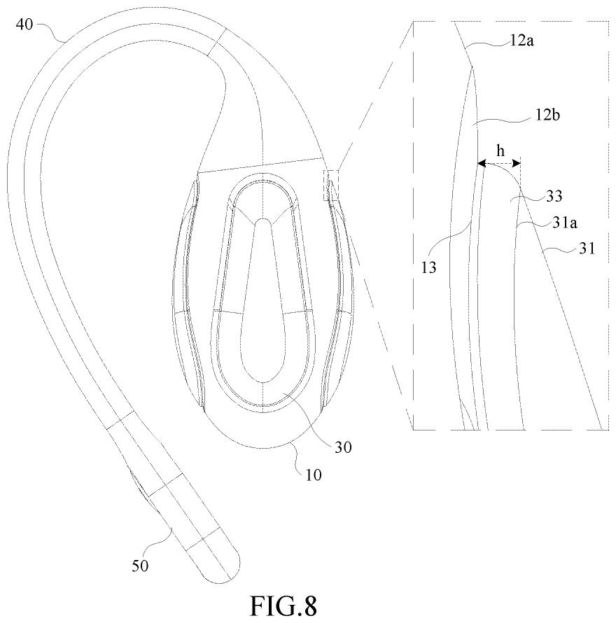

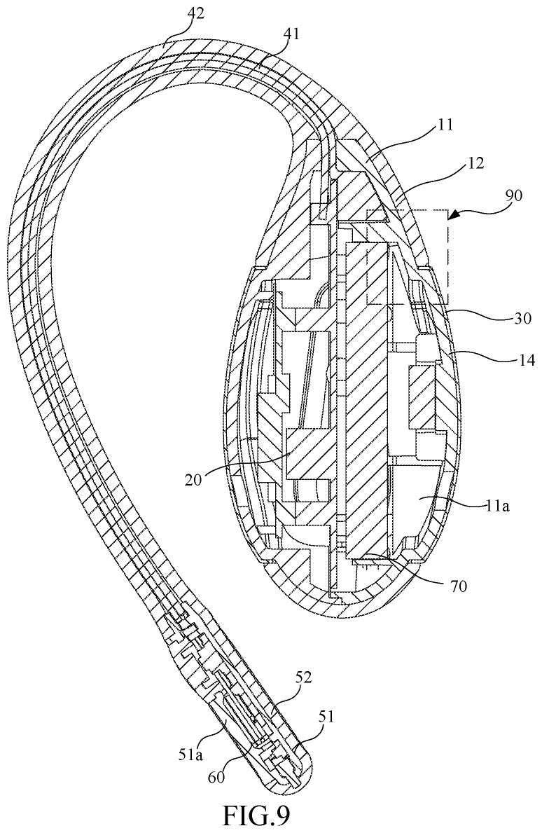

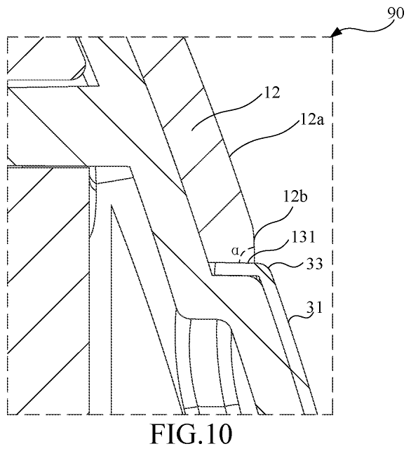

The embodiments of the present application are described in detail below with reference to FIGS. 1-10. FIG. 1 is a schematic structural diagram of a pelvic floor muscle rehabilitation device according to an embodiment of the present application, from a first viewing angle; FIG. 2 is a schematic diagram of a first electrode arrangement of the pelvic floor muscle rehabilitation device according to an embodiment of the present application, from a second viewing angle; FIG. 3 is a schematic diagram of the first electrode arrangement of the pelvic floor muscle rehabilitation device according to an embodiment of the present application, from a third viewing angle; FIG. 4 is a schematic diagram of a second electrode arrangement of the pelvic floor muscle rehabilitation device according to an embodiment of the present application, from the second viewing angle; FIG. 5 is a schematic diagram of the second electrode arrangement of the pelvic floor muscle rehabilitation device according to an embodiment of the present application, from the third viewing angle; FIG. 6 is an exploded structural diagram showing a soft layer of the pelvic floor muscle rehabilitation device and other parts of the pelvic floor muscle rehabilitation device according to an embodiment of the present application; FIG. 7 is an exploded structural diagram of a first frame of the pelvic floor muscle rehabilitation device according to an embodiment of the present application; FIG. 8 is a schematic structural diagram of the pelvic floor muscle rehabilitation device according to an embodiment of the present application, from a fourth viewing angle; FIG. 9 is a schematic cross-sectional structural diagram of the pelvic floor muscle rehabilitation device according to an embodiment of the present application; FIG. 10 is an enlarged view of a partial structure within the dashed box 90 of FIG. 9.

As shown in FIGS. 1-5, one embodiment of the present application provides a pelvic floor muscle rehabilitation device 100, which includes an inner arm 10, an outer arm 50, and a connecting portion 40 connected therebetween. The inner arm 10 is configured to be inserted into a user's vagina during use. The outer arm 50 is configured to be held and operated by a user's single hand outside the vagina, so as to adjust the orientation of the inner arm 10 inside the body. The connecting portion 40 is connected between the first end of the inner arm 10 and the outer arm 50, so that the entire rehabilitation device forms an ergonomic structure.

In one embodiment, referring to FIGS. 1-5, from the first end 15 to the second end 16 of the inner arm 10, the outer cross-sectional diameter first gradually increases and then gradually decreases. The first end 15 is the end proximate to the connecting portion 40, and the second end 16 is the end distal from the connecting portion 40 and is the end that is inserted first during use. This configuration makes the inner arm 10 substantially ellipsoidal as a whole, with its outer diameter first increasing and then decreasing, thereby defining a maximum outer diameter portion 17 on the inner arm 10. This design conforms to human physiological structure, facilitating smooth and comfortable insertion of the inner arm 10 and improving the comfort of the insertion process, while also ensuring that, when inside the body, the maximum outer diameter portion 17 makes good contact with the pelvic floor muscles. Substantially ellipsoidal refers to a profile having a continuously increasing diameter and subsequently decreasing diameter along the longitudinal direction.

As shown in FIGS. 2-5, the inner arm 10 is provided with at least two stimulation electrodes 181, 182 and at least two detection electrodes 191, 192, all of which serve as specific functional types of electrode pads 30. In one specific example, the total number of electrode pads 30 may be four, which may be evenly spaced apart along the circumferential direction of the inner arm 10. Among them, two are stimulation electrodes 181, 182 for delivering electrical stimulation currents to the user; and two are detection electrodes 191, 192 for collecting the user's pelvic floor muscle surface myoelectric signals. This arrangement avoids the need for frequent manual rotation of the device to stimulate pelvic floor muscles in different orientations, as required in the prior art.

Two circumferential arrangement modes of the stimulation electrodes 181, 182 and the detection electrodes 191, 192 are described below.

As shown in FIGS. 2 and 3, in one embodiment, the two stimulation electrodes 181, 182 and the two detection electrodes 191, 192, i.e., the four electrode pads 30, may be alternately arranged along the circumferential direction of the inner arm 10. Optionally, the two stimulation electrodes 181, 182 are respectively located on opposite sides of the inner arm 10, and the two detection electrodes 191, 192 are respectively located on opposite sides of the inner arm 10.

As shown in FIGS. 4 and 5, in another alternative arrangement, the two stimulation electrodes 181, 182 are arranged adjacent to each other, and the two detection electrodes 191, 192 are arranged adjacent to each other.

Regardless of which arrangement is adopted, each electrode pad 30 is an arc-shaped sheet structure extending along the surface of the inner arm 10 from the second end 16 toward the first end 15, and each electrode pad 30 extends at least across the maximum outer diameter portion 17 of the inner arm 10, so as to ensure that after the inner arm 10 is inserted into the vagina, the electrodes can make adequate and stable contact with the pelvic floor muscles.

As shown in FIGS. 2, 3, 4, and 5, in one embodiment, the detection electrodes 191 or 192 are disposed on a side 123 of the inner arm 10 facing toward the connecting portion 40, and no stimulation electrode 181 or 182 is disposed on the side 123 of the inner arm 10 facing toward the connecting portion 40. In this manner, comfort during wearing and electrical stimulation can be increased, improving user experience. The side 123 of the inner arm 10 facing toward the connecting portion 40 may be the side of the inner arm 10 that is close to or faces the connecting portion 40.

As shown in FIG. 1, the outer diameter of the pelvic floor muscle rehabilitation device 100 gradually decreases in the direction from the inner arm 10 to the connecting portion 40, so that the outer surface transitions smoothly from the inner arm 10 to the connecting portion 40, facilitating complete insertion of the inner arm 10 and preventing the connecting portion 40 from excessively stretching the vaginal opening, thereby helping to improve user comfort.

As shown in FIG. 1, in one embodiment, the connecting portion 40 is an arcuate bent section so that a predetermined angle is formed between the inner arm 10 and the outer arm 50, making the product easy to secure to the body and conforming to ergonomic wearing requirements.

In another embodiment, the connecting portion 40 is elongate and deformable by bending. By bending the connecting portion 40, the relative position between the inner arm 10 and the outer arm 50 can be adjusted by the user, for example, bringing them closer to each other or aligning them in a straight line. This deformable feature allows the user to adjust the position and angle of the inner arm 10 within the vagina during treatment, improving the convenience of the therapy. It will be appreciated that the connecting portion 40 may have both arcuate bent and deformable characteristics, or only one of them.

As shown in FIGS. 2, 3, 6, 7, and 9, the inner arm 10 includes a first frame 11 and a first soft layer 12 covering an outer surface of the first frame 11. A first accommodating space 11a is formed inside the first frame 11. A first circuit board 20 is disposed in the first accommodating space 11a, configured to receive myoelectric signals from the detection electrodes 191, 192 and to output electrical stimulation signals through the stimulation electrodes 181, 182. Meanwhile, the pelvic floor muscle rehabilitation device 100 may further include a battery 70, which is also disposed in the first accommodating space 11a and electrically connected to the first circuit board 20, for supplying power to the first circuit board 20, achieving integration and portability of the device.

As shown in FIG. 6, the first soft layer 12 is made of a skin-friendly soft material such as medical-grade silicone or medical-grade rubber, and has a hardness less than that of the first frame 11. This makes the first soft layer 12 softer and more skin-friendly to the touch, allowing the inner arm 10 to better conform to the user's pelvic floor muscles, effectively reducing the foreign body sensation inside the user and improving comfort. The first frame 11 is made of a rigid plastic material.

As shown in FIGS. 2, 3, 6, 7, and 9, with regard to the specific configuration of the frame, in the direction from the first end 15 to the second end 16, the outer diameter of the first frame 11 first gradually increases and then gradually decreases. A circumferential outer surface of the first frame 11 about its axis is provided with outwardly protruding protrusions 14. The electrode pads 30 are disposed to cover the protrusions 14. This outwardly protruding structure enables the electrode pads 30, when the inner arm 10 is inserted into the vagina, to effectively squeeze vaginal secretions outward away from the electrode pads 30, allowing the secretions to disperse, thereby reducing the contact resistance between the electrode pads 30 and the pelvic floor muscles, and thus improving the reliability of the transmission path for electrical stimulation currents or myoelectric signals.

As shown in FIGS. 2, 3, 6, 7, and 9, to achieve reliable fixation and sealing of the electrodes, the first soft layer 12 is provided with openings 13. Each protrusion 14 includes an outwardly protruding top surface 141 and a side surface 142 disposed around a periphery of the top surface 141, the top surface 141 and the side surface 142 being connected via a connecting curved surface 143. Each electrode pad 30 includes a main body portion 31 and a folded edge 32. The shape of the main body portion 31 matches the shape of the top surface 141 of the protrusion 14, and the main body portion 31 covers the top surface 141. The main body portion 31 is exposed through the opening 13 and protrudes beyond an outer surface 12a of the first soft layer 12.

As shown in FIGS. 6, 7, 8, 9, and 10, the folded edge 32 is connected to a periphery 31a of the main body portion 31 and is located on an inner side of the main body portion 31 proximate to the protrusion 14. The folded edge 32 covers the side surface 142 of the protrusion 14, and an outer surface of the folded edge 32 facing away from the protrusion 14 is bonded to an inner sidewall 131 of the opening 13, thereby sealing the opening 13 to prevent ingress of secretions or liquids.

The electrode pad 30 is further formed with a transition curved surface 33 matching the connecting curved surface 143. The folded edge 32 is smoothly connected to the periphery 31a of the main body portion 31 via the transition curved surface 33. After assembly, not only is the main body portion 31 exposed through the opening 13, but the transition curved surface 33 may also be at least partially exposed through the opening 13, such that the exposed portion of the electrode pad 30 and the first soft layer 12 are joined via the smooth transition curved surface 33, avoiding scratching the user and allowing the main body portion 31 of the electrode to more effectively push away secretions when inside the body, further reducing contact resistance.

Regarding the structural dimensions of the electrode pad 30, in one embodiment, referring to FIG. 8, the shortest distance h between the periphery 31a of the main body portion 31 and an edge of the folded edge 32 proximate to the main body portion 31 ranges from 0.2 mm to 2 mm (inclusive). This distance defines the extent of the transition curved surface 33 along the inward-outward direction, ensuring that the main body portion can adequately contact the muscle while also providing a smooth, stepless transition between the folded edge 32 and the main body portion 31, thereby improving user comfort.

To enhance bonding strength, referring to FIG. 6, an outer surface of the folded edge 32 may be provided with an infiltration portion recessed toward an inner surface of the folded edge 32. The infiltration portion is configured to receive an adhesive material, which, after curing, forms a seamless mechanical interlocking structure between the folded edge 32 and the inner sidewall 131 of the opening 13. The infiltration portion may be embodied as a plurality of infiltration holes spaced apart along the circumferential direction of the folded edge 32, or as an infiltration groove located on the folded edge 32 at a position away from the periphery 31a of the main body portion 31, or a combination thereof.

Regarding the configuration of the soft layer at the opening, an edge of the opening 13 is lower than the outer surface 12a of the first soft layer 12. The first soft layer 12 includes a transition bevel 12b disposed around the opening 13, the transition bevel 12b connecting between the outer surface 12a of the first soft layer 12 and the edge of the opening 13. In the exposure direction of the electrode pad 30, the transition bevel 12b gradually moves away from the center of the opening 13. This structure not only facilitates sliding of the electrode pad 30 into the opening 13 during assembly, but also makes the angle between the transition bevel 12b and the inner sidewall 131 of the opening 13 more obtuse, increasing the strength of the opening edge and preventing tearing.

To further stabilize the electrodes, in one embodiment, referring to FIGS. 1 and 6 together, the first soft layer 12 may be divided into a first region 121 relatively close to the opening 13 and a second region 122 relatively far from the opening 13. The first region 121 has an elastic modulus greater than that of the second region 122. This makes the first region 121 less prone to deformation, enabling it to firmly clamp the electrode pad 30 in position when the electrode pad 30 is repeatedly subjected to force, thereby ensuring long-term stability of use.

In one embodiment, from an external perspective, the electrode pad 30 is an elongate flat structure extending in a direction from the first end 15 to the second end 16, for example, having an insole-like or elliptical shape.

To achieve integrated control and signal transmission, the outer arm 50 includes a second frame 51, and a second accommodating space 51a is formed inside the second frame 51. The connecting portion 40 includes a connecting wire 41. The pelvic floor muscle rehabilitation device 100 further includes a second circuit board 60 disposed in the second accommodating space 51a, configured to generate control signals (e.g., power on/off, mode selection, etc.) based on user operations. The second circuit board 60 is electrically connected to the first circuit board 20 in the first accommodating space 11a of the inner arm 10 via the connecting wire 41, so as to transmit the control signals to the first circuit board 20, thereby controlling the operating state of the first circuit board 20. The second frame 51 is made of a rigid plastic material. The second soft layer 52 has a hardness less than that of the second frame 51.

Regarding the flexible covering structure, the outer arm 50 further includes a second soft layer 52 covering an outer surface of the second frame 51. The connecting portion 40 further includes a third soft layer 42 covering the connecting wire 41. The third soft layer 42 is connected between the first soft layer 12 and the second soft layer 52. In a preferred embodiment, the first soft layer 12, the second soft layer 52, and the third soft layer 42 are formed as an integrally molded structure using a single or two-shot injection molding process, such that the entire rehabilitation device has a seamless, smooth transition on the outer surface from the inner arm 10, through the connecting portion 40, to the outer arm 50. This not only provides excellent waterproof performance but also facilitates cleaning, better meeting household hygiene and portability requirements.

In the embodiments of the present application, by configuring the outer cross-sectional diameter of the inner arm to first increase and then decrease from the first end toward the second end to form a maximum outer diameter portion, the inner arm as a whole has an ellipsoidal shape that conforms to human physiological structure. This not only facilitates smooth and stable insertion of the inner arm into the vagina without damaging the vaginal mucosa, but also achieves good conformity with the pelvic floor muscles after insertion, significantly improving user comfort. Meanwhile, by arranging the stimulation electrodes and the detection electrodes spaced apart along the circumferential direction of the inner arm, and having each electrode extend in the direction from the second end toward the first end and cover at least the maximum outer diameter portion, after the inner arm is inserted into the vagina, the electrodes can simultaneously make adequate and stable contact with the pelvic floor muscles at different circumferential locations within the vagina, eliminating the need for the user to frequently manually rotate the device during treatment to adjust electrode orientation. This circumferential multi-directional electrode layout, together with the moderate expansion effect of the maximum outer diameter portion on the vaginal tissue, can reduce the contact resistance between the electrodes and the tissue, facilitate efficient transmission of electrical stimulation currents and accurate acquisition of myoelectric signals, thereby improving the repair effect and detection accuracy for the pelvic floor muscles.

To make the objectives, technical solutions, and advantages of the present application clearer, the following further describes the present application in detail with reference to the accompanying drawings and embodiments. It should be understood that the specific embodiments described herein are merely intended to explain the present application and are not intended to limit the present application.

The above descriptions are only specific implementations of the present application. It should be noted that, for those of ordinary skill in the art, various modifications and refinements can be made without departing from the principles of the present application. These modifications and refinements should also be regarded as falling within the protection scope of the present application.

What is claimed is:

1. A pelvic floor muscle rehabilitation device, comprising:
an inner arm configured to be inserted into a vagina;
an outer arm configured to be held by a user outside the vagina;
a connecting portion connecting the inner arm and the outer arm; and
at least two stimulation electrodes and at least two detection electrodes disposed on the inner arm and spaced apart along a circumferential direction of the inner arm;
wherein the inner arm has a first end proximate to the connecting portion and a second end distal from the connecting portion, an outer cross-sectional diameter of the inner arm first increases and then decreases from the first end toward the second end, thereby forming a maximum outer diameter portion on the inner arm;
and wherein each of the at least two stimulation electrodes and each of the at least two detection electrodes extend in a direction from the second end toward the first end and extend at least across the maximum outer diameter portion.

2. The device of claim 1, wherein a total number of the at least two stimulation electrodes and the at least two detection electrodes is four.

3. The device of claim 2, wherein the stimulation electrodes and the detection electrodes are alternately arranged along the circumferential direction of the inner arm.

4. The device of claim 2, wherein the stimulation electrodes are arranged adjacent to each other, and the detection electrodes are arranged adjacent to each other.

5. The device of claim 1, wherein the inner arm is substantially ellipsoidal in shape.

6. The device of claim 1, wherein each of the at least two stimulation electrodes and the at least two detection electrodes is an arc-shaped sheet electrode extending along a surface of the inner arm.

7. The device of claim 1, wherein the outer arm has an elongated shape and is configured to be held by a single hand to adjust an orientation of the inner arm.

8. The device of claim 1, wherein the connecting portion is an arcuate bent section so that a predetermined angle is formed between the inner arm and the outer arm.

9. The device of claim 1, wherein the connecting portion is elongate and deformable by bending.

10. The device of claim 1, wherein a side of the inner arm facing toward the connecting portion is provided with the detection electrodes, and no stimulation electrode is disposed on the side of the inner arm facing toward the connecting portion.

11. A pelvic floor muscle rehabilitation device, comprising:
an inner arm configured to be inserted into a vagina, the inner arm comprises a first frame and a first soft layer covering an outer surface of the first frame, a first accommodating space being formed inside the first frame;
an outer arm configured to be held by a user outside the vagina;
a connecting portion connecting the inner arm and the outer arm, wherein the inner arm has a first end proximate to the connecting portion and a second end distal from the connecting portion, an outer cross-sectional diameter of the inner arm first increases and then decreases from the first end toward the second end, thereby forming a maximum outer diameter portion on the inner arm;
at least two stimulation electrodes and at least two detection electrodes spaced apart along a circumferential direction of the inner arm, each of the at least two stimulation electrodes and each of the at least two detection electrodes extending in a direction from the second end toward the first end and at least across the maximum outer diameter portion; and
a first circuit board disposed in the first accommodating space, configured to receive myoelectric signals from the detection electrodes and output electrical stimulation signals through the stimulation electrodes; wherein each of the at least two stimulation electrodes and each of the at least two detection electrodes, as electrode pads, are disposed on an outer surface of the first frame and electrically connected to the first circuit board; the first soft layer is provided with openings, the electrode pads being at least partially exposed through the openings, and the first soft layer has a hardness less than a hardness of the first frame.

12. The device of claim 11, wherein in a direction from the first end to the second end, an outer diameter of the first frame first gradually increases and then gradually decreases, a circumferential outer surface of the first frame is provided with outwardly protruding protrusions, and the electrode pads cover the protrusions.

13. The device of claim 12, wherein each protrusion comprises an outwardly protruding top surface, each electrode pad comprises a main body portion, a shape of the main body portion matches a shape of the top surface of the protrusion, the main body portion covers the top surface of the protrusion, and is exposed through the opening and protrudes beyond an outer surface of the first soft layer.

14. The device of claim 13, wherein each protrusion further comprises a side surface disposed around a periphery of the top surface of the protrusion, each electrode pad further comprises a folded edge connected to a periphery of the main body portion and located on an inner side of the main body portion proximate to the protrusion, the folded edge covers the side surface of the protrusion, and an outer surface of the folded edge facing away from the protrusion is bonded to an inner sidewall of the opening.

15. The device of claim 14, wherein the top surface and the side surface of the protrusion are connected via a connecting curved surface, and the electrode pad further comprises a transition curved surface matching the connecting curved surface, the folded edge being connected to the periphery of the main body portion via the transition curved surface.

16. The device of claim 11, wherein an edge of each opening is lower than an outer surface of the first soft layer, the first soft layer comprises a transition bevel disposed around each opening, the transition bevel connects between the outer surface of the first soft layer and the edge of the opening, and in an exposure direction of the electrode pad, the transition bevel gradually moves away from a center of the opening.

17. The device of claim 11, wherein the first soft layer comprises a first region and a second region, the first region being closer to the opening than the second region, and the first region has an elastic modulus greater than that of the second region.

18. The device of claim 11, further comprising a battery disposed in the first accommodating space and electrically connected to the first circuit board.

19. A pelvic floor muscle rehabilitation device, comprising:
an inner arm configured to be inserted into a vagina, the inner arm being provided with stimulation electrodes and detection electrodes;
an outer arm configured to be held by a user outside the vagina;
a connecting portion connecting the inner arm and the outer arm;
a first circuit board disposed inside the inner arm and electrically connected to the stimulation electrodes and the detection electrodes;
a second circuit board disposed inside the outer arm and configured to generate control signals;
a connecting wire extending through the connecting portion, the second circuit board being electrically connected to the first circuit board via the connecting wire, such that the control signals generated by the second circuit board can be transmitted to the first circuit board through the connecting wire.

20. The device of claim 19, wherein an outer surface of the inner arm is covered with a first soft layer, an outer surface of the outer arm is covered with a second soft layer, an outer surface of the connecting portion is covered with a third soft layer, the third soft layer is connected between the first soft layer and the second soft layer, and the first soft layer, the second soft layer, and the third soft layer form an integrally molded structure.
